(19) United States
(12) Patent Application Publication
(10) Pub. No.: US 2007/0270793 A1
(43) Pub. Date: Nov. 22, 2007

(54) Methods for endocardial ablation

(76) Inventor: Omar M. Lattouf, (US)

Correspondence Address: LAW OFFICE OF COLLEN A. BEARD, LLC, P. O. BOX 1064, DECATUR, GA, 30031-1064, US

(21) Appl. No.: 11/784,681

(22) Filed: Apr. 9, 2007

Related U.S. Application Data
(63) Continuation-in-part of application No. 10/313,198, filed on Dec. 6, 2002.
Application No. 10/313,198 is a continuation-in-part of application No. 10/295,390, filed on Nov. 15, 2002, now Pat. No. 6,978,176.
(60) Provisional application No. 60/340,062, filed on Dec. 8, 2001.
(60) Provisional application No. 60/365,918, filed on Mar. 20, 2002.
(60) Provisional application No. 60/369,988, filed on Apr. 4, 2002.

(51) Int. Cl. A61B 18/18

(52) U.S. Cl. 606/41

(57) ABSTRACT

Methods and devices for performing endocardiac ablation by accessing the interior of the heart through the wall of the heart at its apex.

### CROSS REFERENCE TO RELATED APPLICATIONS

This application is a continuation in part of application Ser. No. 10/313,198, filed on Dec. 6, 2002 which is a continuation-in-part of application Ser. No. 10/295,390, filed on Nov. 15, 2002 which is related to and claims the priority of provisional application Ser. No. 60/340,062, filed Dec. 8, 2001, provisional application Ser. No. 60/365,918, filed Mar. 20, 2002, and provisional application Ser. No. 60/369,988, filed Apr. 4, 2002. The entire contents of these applications are incorporated herein by reference.

### BACKGROUND OF THE INVENTION

The invention is in the field of cardiac health, more specifically in the field of minimally invasive methods for cardiac ablation.

Heart muscle contractions are controlled by electricity flowing throughout the heart. Normally, this electrical flow is in a regular, measured pattern. Sometimes, however, the electrical flow gets blocked or travels the same pathways repeatedly, creating something of a “short circuit” that disturbs normal heart rhythms. Medicine often helps this condition. In some cases, however, the most effective treatment is to physically destroy the tissue where the short circuit occurs. The procedure to destroy this tissue is called cardiac ablation.

Ablation can be conducted using different types of energy sources such as radiofrequency (RF), microwave, laser, ultrasound, radiation using a beta source, and cryothermy (cold temperatures). The energy is delivered via an instrument, such as a catheter, which is inserted into the body and the tip of which is placed at the site to be ablated. The instrument may have a shaped tip to cover a greater surface area. Inflatable balloons to deliver the energy have also been developed, which also allow for ablation of a greater surface area.

Cardiac ablation can be performed inside the heart (endocardial) or on the outside of the heart (epicardial). The location of the ablation performed depends upon the type of arrhythmia and the presence of other heart disease.

Epicardial ablation is often performed using traditional “open” surgery, where the patient's chest is opened up to allow access to the heart. Epicardial ablation is thus easy in one respect—the site to be treated can be relatively easily accessed. Endocardial ablation on the other hand has the additional complication of requiring access to the interior of the heart. Endocardial ablation is also commonly performed using traditional open surgery but endocardial ablation is also conducted using percutaneous access. During this procedure a catheter is inserted at the femoral or carotid artery and threaded into a specific area of the heart. Travel of the catheter is monitored using a fluoroscope. This procedure is often called percutaneous cardiac ablation.

Ablation is used in all areas of the heart. Most often, cardiac ablation is used to treat rapid heartbeats that begin in the upper chambers, or atria, of the heart. As a group, these are known as supraventricular tachycardias, or SVTs. Types of SVTs are atrial fibrillation and atrial flutter, AV nodal reentry tachycardia (AVNRT), AV reentrant tachycardia, and atrial tachycardia. Cardiac ablation can also be used to treat accessory pathways tachycardia which is a rapid heart rate due to an extra abnormal pathway or connection between the atria and the ventricles. The impulses travel through the extra pathways as well as through the usual route. This allows the impulses to travel around the heart very quickly, causing the heart to beat unusually fast.

Less frequently, ablation is used to treat heart rhythm disorders that begin in the heart's lower chambers, known as the ventricles. Ventricular tachycardia (V-tach) is a rapid heart rhythm originating from the ventricles of the heart. The rapid rate prevents the heart from filling adequately with blood; therefore, less blood is able to pump through the body. This can be a serious arrhythmia, especially in people with heart disease.

In all types of ablation procedures, once the catheter or other device reaches the heart, electrodes at the tip of the catheter gather data and a variety of electrical measurements are made. The data pinpoints the location of the faulty electrical site. Once the damaged site is confirmed, the ablation energy is delivered to destroy a small amount of tissue, hopefully ending the disturbance of electrical flow through the heart and restoring a healthy heart rhythm.

Percutaneous cardiac ablation has several issues that make it less than desirable. For one thing, the catheters that are used for percutaneous cardiac ablation are limited in size because they must be threaded through the vasculature into the heart. This means that the area of tissue that can be ablated is very small and the tip must be maneuvered around quite a bit if the area to be treated is large. In cases where more than one type of tool is used, each tool must be threaded separately, adding to the length of the process.

Maneuverability of a catheter which is threaded such a long distance is limited, which means that it is difficult and sometimes impossible to locate the catheter electrode tip exactly where the cardiac tissue needs to be ablated. This adds to the total length of the procedure. It also means that the ablation is sometimes less effective than required. Another issue with percutaneous access can be various vascular complications such as bleeding, dissection, and rupture of a blood vessel. Moreover, some areas of the heart are difficult to access percutaneously.

Accordingly, to avoid the disadvantages of percutaneous cardiac ablation, the present invention provides a method for endocardial ablation using minimally invasive access to the interior of the heart. The area of the heart that is accessed is the apical area of the heart, which is the rounded inferior extremity of the heart formed by the left and right ventricles. In normal healthy humans it generally lies beneath the fifth left intercostal space from the mid-sternal line.

Access to the interior of the heart via the apex (trans-apical access) is taught in U.S. Pat. No. 6,978,176 to Lattouf. This patent is primarily directed to mitral valve repair but the method taught therein is also described as being useful for ablation.

Endocardial ablation using trans-apical delivery is advantageous in that it is not limited by the space that is available within the vasculature. The instruments that are used are shorter than those used for percutaneous access, meaning they can be maneuvered more easily and accurately.

### SUMMARY OF THE INVENTION

The present invention is directed to methods and devices for performing endocardiac ablation. The methods rely upon access to the interior of the heart through the wall of the heart at its apex.

The procedure generally includes first gaining access to the patient's chest cavity through the chest cavity. The patient's heart wall is pierced to provide a passageway through the heart wall to the left or right ventricle. The passageway is formed through a region of the heart wall at or near the apex of the patient's heart.

In one embodiment, an instrument port is installed in the ventricular wall passageway. The port is configured to enable passage of instruments for the procedure through the heart wall into the heart chamber while preventing loss of blood through the passageway. An instrument port is not required but is preferred in some respects.

The ablation catheter or other ablation tool is then passed through the instrument port or passageway and into the area to be treated, such as the left atrium. In one embodiment, an instrument guide is used, to provide a guide for the ablation tool. Once the ablation procedure is completed, the instruments are withdrawn through the instrument port (if one is used) and then the opening in the patient's chest. The instrument port may be left in place or the port may be removed and the passageway sutured or otherwise suitably closed.

The invention will become more apparent from the following detailed description and accompanying exemplary drawings.

### BRIEF DESCRIPTION OF THE DRAWINGS

FIG. 1 is a perspective view of a patient's chest, partially illustrating the location of the patient's heart within the chest cavity, with part of the heart wall removed to expose the left ventricular and atrial chambers and showing the method of the present invention for ablation in the left atrium.

FIG. 2 is an elevational view of an ablation instrument of the invention.

### DETAILED DESCRIPTION OF THE INVENTION

The present method can be used with ablation devices that deliver various types of energy, such as radiofrequency (RF), microwave, laser, ultrasound, radiation using a beta source, and cryothermy (cold temperatures). Some commercially available devices may be suitable for use in the invention. However, devices used for percutaneous ablation are not entirely suitable because they are longer and more flexible than is desirable. Devices used for open heart ablation may not be suitable because they may be larger than is desired for insertion through the chest trocar and the optional heart wall port. However, in general, the various types of ablation instruments that are currently used, and that are anticipated for use, can be modified for use in the invention.

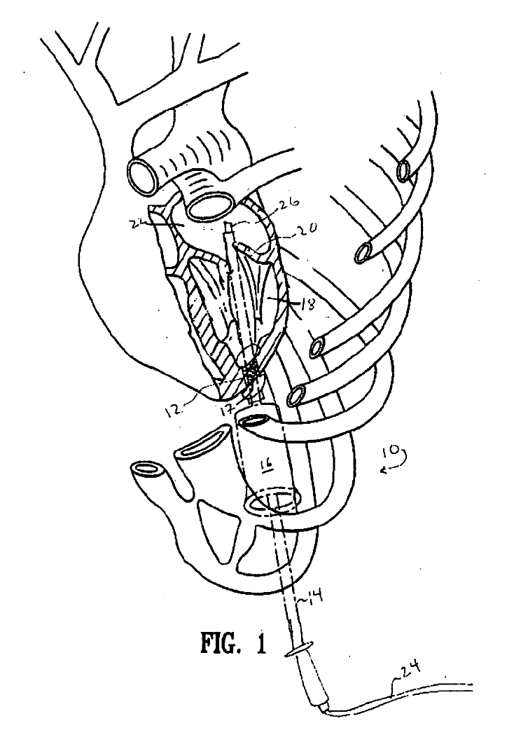

FIG. 1 demonstrates the method of the invention for ablation in the left atrium. In this embodiment, an instrument port 12 is implanted at the apex 17 of the left ventricle. Instrument guide 14 is inserted through chest trocar 16, through the instrument port 12, into the left ventricle 18, past the mitral valve 20, and into the left atrium 22. Ablation catheter 24 is threaded through the instrument guide 14 so that its tip 26 is in the left atrium.

The chest trocar 12 can be one that is commercially available, such as those available from U.S. Surgical and others. It is placed in a manner known to those skilled in the art, preferably though an intercostal space between two of the patient's ribs.

To the extent required, the patient's deflated lung is moved out of the way, and then the pericardium on the patient's heart wall is removed to expose a region of the epicardium at the heart apex. The patient's heart wall is pierced at the exposed epicardial location using a piercing element such as a 14 gauge needle. A guide wire is advanced through the inner lumen of the needle into the heart chamber to the area of the heart to be treated, such as the left atrium. The penetrating needle may then be removed leaving the guide wire in place.

Using a series of dilators placed over the guidewire, a hole is formed large enough for insertion of the instrument port 14. The port is configured to enable passage of instruments for the procedure through the heart wall into the heart chamber while preventing loss of blood through the passageway and preferably includes a one-way valve.

The port can be the port disclosed in U.S. Pat. No. 6,978,176 to Lattouf. Other ports that can be used are disclosed in U.S. Publication No. 2006/0074484 to Huber and U.S. Publication No. 2007/0027534 to Bergheim et al. Especially preferred instrument ports are the ones described in U.S. patent application Ser. No. ______ to Lattouf et al., filed on Apr. 6, 2007 as Express mail # EQ230030562US.

After the instrument port 14 is placed into position, in the left ventricular apex as shown in FIG. 1, the instrument for performing the ablation procedure (such as ablation catheter 24) is passed through the instrument port 14. As shown in FIG. 1, the ablation catheter 24 is inserted through the port 12, into the left ventricle 18, through the mitral valve 20, and into the left atrium 22.

In one embodiment, an instrument guide is inserted through the instrument port, or through the heart wall if a port is not used, to provide a guide for the ablation tool. The instrument guide is a cylindrical, stiff tube with desirably a steerable tip to steer the ablation tool functional head to the desired location. An especially preferred instrument guide is the one described in U.S. patent application Ser. No. ______ to Lattouf et al., filed on Apr. 6, 2007 as Express mail # EQ230030562US.

While an instrument port and an instrument guide are used in the preferred embodiment of the method, they are not required. The heart wall can be pierced as described above and the hole enlarged as described above. The ablation tool can simply be inserted through the enlarged hole and the tip moved to the desired location. If a percutaneous ablation catheter is used, the instrument guide is especially helpful to provide support to the catheter.

Examples of percutaneous access catheters which can be used are the Livewire TC ablation catheters (RF energy) and Epicor cardiac ablation system (high intensity focused ultrasound), both offered by St. Jude Medical, and the Artic Circler circular cryocatheter offered by Cryocath Inc. Many other percutaneous access ablation catheters can also be used.

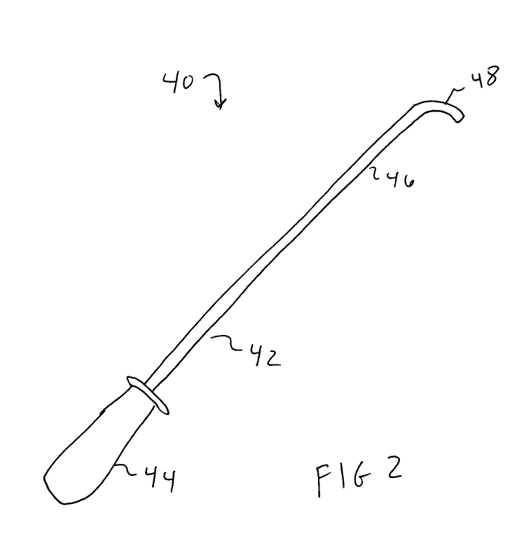

In one embodiment, a specially designed ablation tool is used which is stiff enough to be inserted through the heart wall and guided to the desired location. FIG. 2 shows this ablation tool 40 in more detail. The ablation tool 40 has a relatively stiff body portion compared to percutaneous access catheters and is relatively short compared to percutaneous access catheters. Desirably the ablation tool is about 5 to 25 cm in length, more desirably about 8 to 18 cm. The proximal end 42 of the ablation tool will extend out of the patient's body and has a handle 44 attached. The distal end 46 includes functional distal tip 48 and desirably is steerable via the handle. Various methods for steering the distal tip of a catheter are known in the art and can be used for the ablation tool 40. Various ablation energies are known in the art and can be used in ablation tool 40. The primary differences between ablation tool 40 and prior art percutaneous ablation catheters are the length and stiffness of the tool 40. These characteristics enable the ablation tool 40 to be used for transapical ablation. The tool can be used without the need for an instrument port or instrument guide (although these can be used if desired as discussed above).

The ablation tool body can be made from a stiff material such as PEBAX® or polystyrene. The body portion is desirably stiff enough to be pushable and maneuverable. The outer diameter of the ablation tool is preferably about 2 to 45 French.

Once the ablation procedure is completed, the instruments for the procedure are withdrawn through the instrument port and then the opening in the patient's chest. The instrument port will close upon instrument removal to eliminate or at least minimize blood leakage through the port. The instrument port may be left in place or the port may be removed and the passageway sutured or otherwise suitably closed.

Modifications and variations of the present invention will be apparent to those skilled in the art from the forgoing detailed description. All modifications and variations are intended to be encompassed by the following claims. All publications, patents, and patent applications cited herein are hereby incorporated by reference in their entirety.

What is claimed is:

1. A method for endocardiac ablation comprising the steps:
piercing the heart wall at its apex;
forming a passageway large enough for passage of an ablation tool;
inserting an ablation tool having a functional end through the passageway into the heart and placing the functional end of the ablation tool at the area to be treated; and
ablating the desired tissue.

2. The method of claim 1, wherein an instrument port having a lumen is implanted in the passageway formed in the heart wall and the ablation tool is inserted through the instrument port into the heart.

3. The method of claim 1, wherein an instrument guide having a lumen therethrough is inserted through the passageway and the ablation tool is inserted through the instrument guide lumen into the heart.

4. The method of claim 2, wherein an instrument guide having a lumen therethrough is inserted through the instrument port lumen and the ablation tool is inserted through the instrument guide lumen into the heart.

5. The method of claim 1, wherein the ablation tool is an ablation catheter designed for percutaneous use.

6. The method of claim 3, wherein the ablation tool is an ablation catheter designed for percutaneous use.

7. The method of claim 4, wherein the ablation tool is an ablation catheter designed for percutaneous access.

8. The method of claim 1, wherein the ablation tool is from about 5 to 25 cm in length.

9. The method of claim 1, wherein the ablation tool delivers energy selected from the group consisting of radiofrequency, microwave, laser, ultrasound, beta radiation, and cryothermy.

10. The method of claim 1, wherein the ablation tool is inserted into the body through a chest trocar.

11. The method of claim 1, wherein the passageway in the heart is in the left ventricular apex.

12. The method of claim 1, wherein the passageway allows passage of ablation tools from about 2 to 45 Fr.

13. The method of claim 1, wherein the method is used to ablate tissue in the left atrium.
